(19) United States
(12) Patent Application Publication
(10) Pub. No.: US 2023/0218664 A1
(43) Pub. Date: Jul. 13, 2023

(54) HEALTH FOOD FOR PROMOTION OF QUALITY OF SLEEPY AND METABOLISM

(71) Applicant: Tian Yu Wang, Kaohsiung City (TW)

(72) Inventor: Tian Yu Wang, Kaohsiung City (TW)

(21) Appl. No.: 17/571,555

(22) Filed: Jan. 10, 2022

(51) Int. Cl. A61K 33/30; A61K 33/06; A61K 33/04; A61K 33/24; A61K 9/00; A61K 31/51; A61K 31/525; A61K 31/455; A61K 31/197; A61K 31/4415; A61K 31/519; A61K 31/714; A61K 31/4188; A61K 36/064; A61K 47/38; A61K 47/36; A61K 47/02; A23L 33/00; A23L 33/16; A23L 33/145; A23L 33/15

(52) U.S. Cl. CPC A61K 33/30; A61K 33/06; A61K 33/04; A61K 33/24; A61K 9/0056; A61K 31/51; A61K 31/525; A61K 31/455; A61K 31/197; A61K 31/4415; A61K 31/519; A61K 31/714; A61K 31/4188; A61K 36/064; A61K 47/38; A61K 47/36; A61K 47/02; A23L 33/40; A23L 33/16; A23L 33/145; A23L 33/15; A23V 2002/00

(57) ABSTRACT

A health food for promotion of quality of sleepy and metabolism, comprises the following components with a weight ratio of: 44.93 Milligrams ±25% of Magnesium (Mg), 3.8 Milligrams (±25%) of Zinc (Zn), 40 Micrograms (±25%) of Selenium (Se) and 40 Micrograms (±25%) of Chromium (Cr). Magnesium has the effect to promote ability of cardiovascular disease prevention; zinc can increase the power of mitochondria and enhance the power of metabolism, selenium serves to reduce risks of suffering from cancers and suppress the probability of infection of RNA virus, and Chromium has the effect of reduction of type 2 diabetes. A combination of above mentioned components has the overall effect of promotion of sleep quality, reduction of demand of sleep and increase abilities of diseases preventions.

### FIELD OF THE INVENTION

The present invention is related to health foods, and in particular to a health food for promotion of quality of sleepy and metabolism.

### BACKGROUND OF THE INVENTION

Health foods means specific foods which have the function of healthy or supplements of nutrition, such as vitamins, minerals, etc. These health foods generally have forms of capsules for sale. Commonly, health foods contain specific components and functions for applying to specific people. Basically, health foods have functions of adjusting functions of bodies and do not aimed at curing diseases. The health foods have no harm to human bodies, and basically are kinds of supplements of nutrition or foods. Health foods are not equal to drugs, which is not aimed to cure disease, but to increase health of people. There are many different forms of health foods and therefore, users select the health foods based on needs of bodies, even they consider convenience, absorption speeds of bodies, or some special needs.

Therefore, object of the present invention is to provide a health food which has the function of promotion of quality of sleepy and metabolism

### SUMMARY OF THE INVENTION

Accordingly, the object of the present invention is to provide a health food for promotion of quality of sleepy and metabolism, wherein the present invention is extracted from natural materials which are easily absorbed by human bodies without any chemical additives and no residues are left in the bodies. Polypeptide carriers can be released. The present invention is healthy and has no harm to the bodies. The capsules of the present invention can promote quality of sleepy and enhance metabolism so that the users are youthful, vigorous and smartful.

To achieve above objects, the present invention provides a health food for promotion of quality of sleepy and metabolism, comprising the following components with a weight ratio of:

Magnesium (Mg) 44.93 Milligrams ±25%;

Zinc (Zn) 3.8 Milligrams (±25%);

Selenium (Se) 40 Micrograms (±25%; and

Chromium (Cr) 40 Micrograms (±25%).

wherein magnesium has the effect to promote ability of cardiovascular disease prevention; zinc can increase the power of mitochondria and enhance the power of metabolism, selenium serves to reduce risks of suffering from cancers and suppress the probability of infection of RNA virus, and Chromium has the effect of reduction of type 2 diabetes; a combination of above mentioned components has the overall effect of promotion of sleep quality, reduction of demand of sleep and increase abilities of diseases preventions.

The above four components are further added with natural yeast extract which consists of the following components with a weight ratio of:

Vitamins B1 1.15 Milligrams (±25%);

Vitamins B 2 1.92 Milligrams (±25%);

Niacin 1.44 Milligrams (±25%);

Pantothenic acid 0.19 Milligrams (±25%);

Vitamins B6 1.92 Milligrams (±25%);

Folic acid 19.2 Micrograms (±25%);

Vitamins B12 0.19 Micrograms (±25%); and

Biotin 1.92 Milligram (±25%)

### BRIEF DESCRIPTION OF THE DRAWINGS

FIG. 1 shows the distribution of the main components of the present invention.

FIG. 2 shows the distribution of the main components and additives of the present invention.

FIG. 3 shows a form of the capsule according to the present invention.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

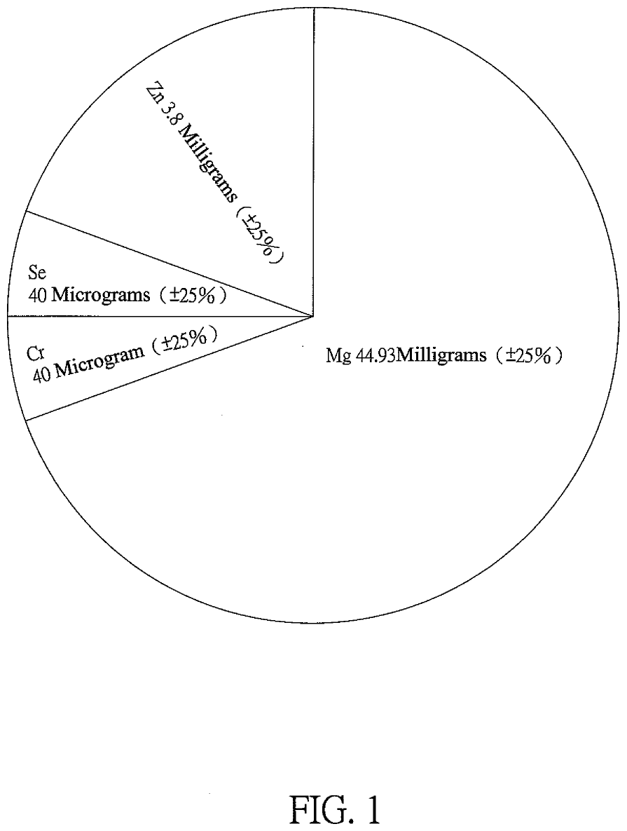

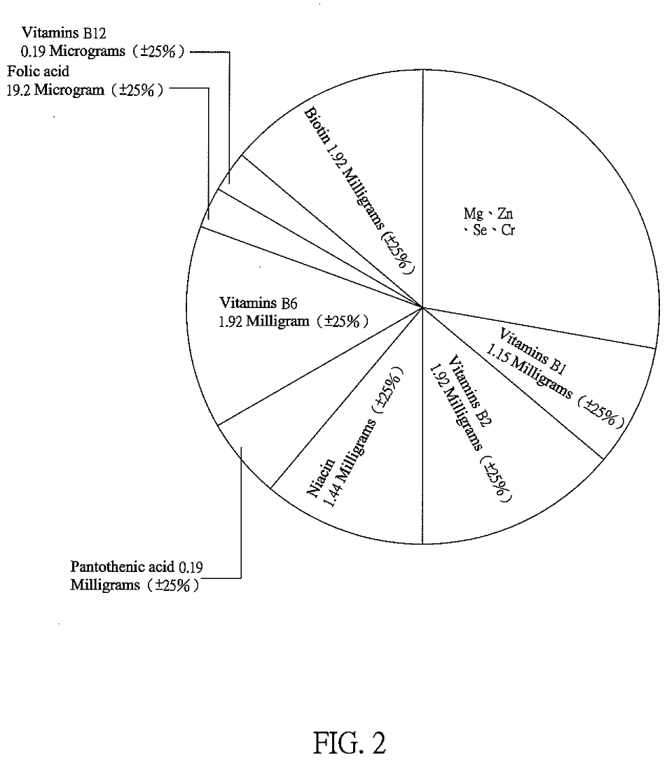

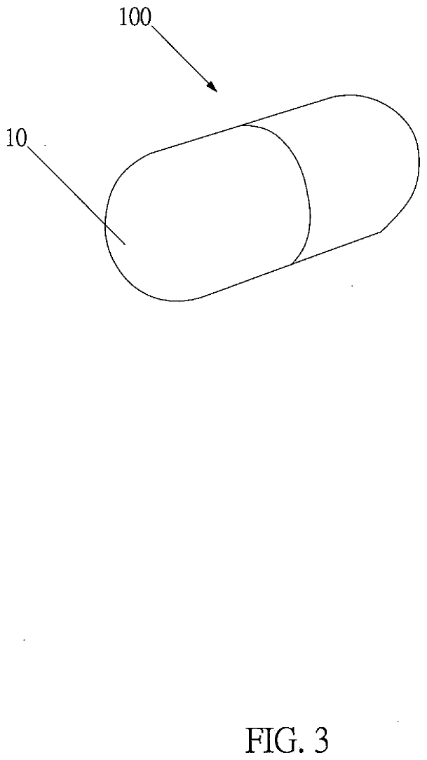

With reference to FIGS. 1 to 3, the components of the present invention are shown. The present invention is related to health foods which are generally existed as capsules 100. In general, a capsule has an elliptical cross sections and an inner space for containing various components therein as illustrated in FIG. 3. A capsule includes the following elements.

A capsule body 10 fills following components:

Magnesium (Mg) 44.93 Milligrams (±25%, it means that the weight of Magnesium is adjustable in the range of ±25% of the weight thereof. This is identical in following description, and thus the details will not be further described)

Zinc (Zn) 3.8 Milligrams (±25%);

Selenium (Se) 40 Micrograms (±25%; and

Chromium (Cr) 40 Micrograms (±25%).

Above elements are primary components of the present invention.

The magnesium has the effect to promote ability of cardiovascular disease prevention. Zinc can increase the power of mitochondria and enhance the power of metabolism. Selenium serves to reduce risks of suffering from cancers and suppress the probability of infection of RNA virus, and Chromium has the effect of reduction of type 2 diabetes. However, the combination of above mentioned components has the overall effect of promotion of sleep quality, reduction of demand of sleep and increase abilities of diseases preventions.

The above four components are further added with natural yeast extract which consists of the following components:

Vitamins B1 1.15 Milligrams (±25%);

Vitamins B 2 1.92 Milligrams (±25%);

Niacin 1.44 Milligrams (±25%);

Pantothenic acid 0.19 Milligrams (±25%);

Vitamins B6 1.92 Milligrams (±25%);

Folic acid 19.2 Micrograms (±25%);

Vitamins B12 0.19 Micrograms (±25%); and

Biotin 1.92 Milligram (±25%)

In production stage, they are formed by following components:

Magnesium citrate, Zinc Yeast (containing 12% of zinc), Selenium yeast (containing 5% of selenium), Chromium Yeast (containing 0.29% of Chromium). The yeast of the Zinc Yeast (containing 12% of zinc), Selenium yeast (containing 5% of selenium), Chromium Yeast are selected from, for example, beer yeast, brewers yeast (Saccharomyces cerevisiae), etc.

In that, ratio of the Magnesium citrate, Zinc Yeast (containing 12% of zinc, Selenium yeast (containing 5% of selenium), Chromium Yeast (containing 0.29% of Chromium) is aimed to cause that after mixing of these yeasts, the mixing powders contain 3.8 Milligrams (±25%) of Zinc (Zn); 40 Micrograms (±25%) of Selenium (Se); and 40 Micrograms (±25%) of Chromium (Cr). These yeasts are full of minerals, and other natural elements which can increase absorption rate of human body.

Moreover, natural yeast extracts are added thereto, which is mainly used to provide above mentioned Vitamins B1, Vitamins B2, Niacin, Pantothenic acid, Vitamins B6, Folic acid, Vitamins B12 and Biotin. The ratio of the natural yeast extracts to other components of the present invention is mainly to cause that after mixing, it contains 1.15 Milligrams (±25%) of Vitamins B1, 1.92 Milligrams (±25%) of Vitamins B 2, 1.44 Milligrams (±25%) of Niacin, 0.19 Milligrams (±25%) of Pantothenic acid, 1.92 Milligrams (±25%) of Vitamins B6, 19.2 Micrograms (±25%) of Folic acid, 0.19 Micrograms (±25%) of Vitamins B12, and 1.92 Milligram (±25%) of Biotin.

In application, the present invention has a form as a capsule. Components of the capsule are adjustable as desired, such as each capsule (each capsule has a weight of 74±10% mg) may further contain Hydroxypropyl Methyl Cellulose (HPMC), purified water, carrageenan, and potassium Chloride, the ratio of which are adjustable as desired.

Other than the capsule shown in FIG. 3, the present invention can be presented as powders.

Advantages of the present invention are that the present invention is extracted from natural materials which are easily absorbed by human bodies without any chemical additives and no residues are left in the bodies. Polypeptide carriers can be released. The present invention is healthy and has no harm to the bodies. The capsules of the present invention can promote quality of sleepy and enhance metabolism so that the users are youthful, vigorous and smartful.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A health food for promotion of quality of sleepy and metabolism, comprising the following components with a weight ratio of:
Magnesium (Mg) 44.93 Milligrams ±25%;
Zinc (Zn) 3.8 Milligrams (±25%);
Selenium (Se) 40 Micrograms (±25%; and
Chromium (Cr) 40 Micrograms (±25%).
wherein magnesium has an effect of promoting ability of cardiovascular disease prevention; zinc can increase the power of mitochondria and enhance the power of metabolism, selenium serves to reduce risks of suffering from cancers and suppress the probability of infection of RNA virus, and Chromium has the effect of reduction of type 2 diabetes; a combination of above mentioned components has the overall effect of promotion of sleep quality, reduction of demand of sleep and increase abilities of diseases preventions.

2. The health food for promotion of quality of sleepy and metabolism as claimed in claim 1, further added with natural yeast extract which consists of the following components with a weight ratio of:
Vitamins B1 1.15 Milligrams (±25%);
Vitamins B 2 1.92 Milligrams (±25%);
Niacin 1.44 Milligrams (±25%);
Pantothenic acid 0.19 Milligrams (±25%);
Vitamins B6 1.92 Milligrams (±25%);
Folic acid 19.2 Micrograms (±25%);
Vitamins B12 0.19 Micrograms (±25%); and
Biotin 1.92 Milligram (±25%)

3. The health food for promotion of quality of sleepy and metabolism as claimed in claim 2, wherein in production stage, natural yeast extract is formed by following components:
Magnesium citrate, Zinc Yeast (containing 12% of zinc), Selenium yeast (containing 5% of selenium), and Chromium Yeast (containing 0.29% of Chromium).

4. The health food for promotion of quality of sleepy and metabolism as claimed in claim 3, wherein the yeasts of the Zinc Yeast (containing 12% of zinc), Selenium yeast (containing 5% of selenium), Chromium Yeast are selected from beer yeast, brewers yeast (Saccharomyces cerevisiae).

5. The health food for promotion of quality of sleepy and metabolism as claimed in claim 1, wherein a ratio of the Zinc Yeast (containing 12% of zinc), Selenium yeast (containing 5% of selenium), Chromium Yeast (containing 0.29% of Chromium) causes that after mixing of the zinc yeast, Selenium yeast and Chromium yeast, the mixing powders contain 3.8 Milligrams (±25%) of Zinc (Zn); 40 Micrograms (±25%) of Selenium (Se); and 40 Micrograms (±25%) of Chromium (Cr).

6. The health food for promotion of quality of sleepy and metabolism as claimed in claim 1, further comprising Hydroxypropyl Methyl Cellulose (HPMC), purified water, carrageenan, and potassium Chloride, a ratio of which are adjustable.
